(19) United States
(12) Patent Application Publication
(10) Pub. No.: US 2025/0341528 A1
(43) Pub. Date: Nov. 6, 2025

(54) USE OF ANTI-CITRULLINATED PEPTIDE ANTIBODY AS BIOMARKER FOR DIAGNOSIS AND PROGNOSIS OF AORTIC VALVE STENOSIS

(71) Applicants: THE ASAN FOUNDATION, Seoul (KR); UNIVERSITY OF ULSAN FOUNDATION FOR INDUSTRY COOPERATION, Ulsan (KR)

(72) Inventors: Sahmin LEE, Seoul (KR); Eunhye JI, Seoul (KR); Ah-Ram KIM, Busan (KR)

(73) Assignees: THE ASAN FOUNDATION, Seoul (KR); UNIVERSITY OF ULSAN FOUNDATION FOR INDUSTRY COOPERATION, Ulsan (KR)

(21) Appl. No.: 18/573,608

(22) PCT Filed: Jun. 22, 2022
(86) PCT No.: PCT/KR2022/008888

§ 371 Date: Dec. 22, 2023

(30) Foreign Application Priority Data
Jun. 23, 2021 (KR) 10-2021-0081578

(51) Int. Cl. G01N 33/68

(52) U.S. Cl. CPC G01N 33/6893; G01N 33/6854; G01N 2440/18; G01N 2800/323; G01N 2800/52

(57) ABSTRACT

The present invention relates to a method for providing information for the diagnosis and/or prognosis of aortic valve stenosis, and was arrived at by discovering that an increase in anti-citrullinated peptide antibody levels is associated with the onset and prognosis of aortic valve stenosis. The present inventors discovered that the accurate early diagnosis of aortic valve stenosis is possible through measuring anti-citrullinated peptide antibody levels, and the degree of aortic valve deformity, severity, and probability of complications such as hypertension, rheumatoid arthritis, and interstitial lung disease can be predicted, and thus the anti-citrullinated peptide antibody is expected to be used as a biomarker for more effective diagnosis, prognosis, and treatment of aortic valve stenosis.

### TECHNICAL FIELD

The present invention relates to a method for providing information for diagnosing and/or predicting prognosis of aortic stenosis.

The present invention claims priority based on Korean Patent Application No. 10-2021-0081578 filed on Jun. 23, 2021. All contents disclosed in the specifications and drawings of these applications are incorporated herein by reference.

### BACKGROUND ART

Aortic stenosis (AS) is a condition where the diameter of the aortic valve narrows, preventing sufficient blood flow from the left ventricle to the aorta. In modern societies with a high proportion of elderly populations, it is the most commonly found valvular heart disease. Aortic stenosis is known to occur due to inflammation resulting from endothelial cell damage caused by mechanical stress, leaflet fibrosis due to lipid penetration, thickening, and calcification. It is accompanied by aortic sclerosis along with calcification of the aortic valve. While many cases of mild aortic stenosis are largely asymptomatic, leading to a lengthy subclinical period, once it progresses to moderate severity or more, blood cannot flow smoothly throughout the body, causing symptoms such as dizziness, chest pain, and syncope, becoming fatal. If aortic stenosis is not treated, the annual mortality rate reaches 25%, with an average survival period of merely 2 to 3 years.

Currently, the severity of aortic stenosis is assessed through echocardiography. However, if the ultrasound image is not clear or cannot be accurately discerned, complex processes are needed, such as supplementing with transesophageal echocardiography, similar to gastrointestinal endoscopy, by inserting an instrument combined with a probe into the esophagus to observe the heart across the esophagus. For a more accurate assessment, catheterization of the heart is required, which involves a complex procedure of locally anesthetizing, inserting a tube through the femoral artery, and using X-ray imaging to measure the pressure and oxygen saturation in various sections of the heart, causing discomfort to the patient. Recently, the diagnosis of aortic stenosis is also made based on the presence of myocardial fibrosis, but it lacks efficiency as it requires a heart biopsy.

Since aortic stenosis is mainly caused by degenerative reasons, accurate diagnosis with the current technology is challenging. Especially, as described above, aortic stenosis is mostly asymptomatic in its early stages, making it difficult to detect, and once it progresses to a severe stage, it becomes life-threatening to the patient. Therefore, there is an urgent need to discover biomarkers for the rapid and accurate diagnosis and prognosis prediction of aortic stenosis.

### DISCLOSURE

### Technical Problem

The present invention has been devised to address the aforementioned problems. The inventors have, upon conducting a retrospective observational study on patients who underwent both echocardiography and anti-citrullinated peptide antibody tests, ascertained that an increase in the titer of the anti-citrullinated peptide antibodies may act as an independent predictor for the progression of aortic stenosis, thereby completing the invention.

Consequently, the present invention is directed to providing a method for providing information for diagnosing or predicting the prognosis of aortic stenosis. Additionally, the present invention is directed to providing a screening method for a therapeutic substance for aortic stenosis.

Moreover, the present invention is directed to providing a composition and a kit comprising the same for diagnosing or predicting the prognosis of aortic stenosis.

However, the technical problem the present invention intends to address is not limited to the problems mentioned above, and other problems not mentioned will be clearly understood by those with ordinary skill in the technical field from the description below.

### Technical Solution

The present invention provides a method for providing information for diagnosing or predicting the prognosis of aortic stenosis, comprising the steps of:
(S1) measuring at least one selected from the group consisting of the level of anti-citrullinated peptide antibodies, the level of citrullinated protein, and the activity degree of citrullinated protein from a biological sample separated from a test subject; and
(S2) confirming whether the level of the anti-citrullinated peptide antibodies, the level of citrullinated protein, or the activity degree of citrullinated protein measured in step (S1) is increased compared to a control group.

In one embodiment of the present invention, the method for providing information may further comprise (S3) a step of diagnosing aortic stenosis or determining that the prognosis of aortic stenosis is unfavorable if the level of anti-citrullinated peptide antibodies, the level of citrullinated proteins, and/or the activity degree of citrullinated proteins measured in step (S1) is increased compared to the control group.

In one embodiment of the present invention, the biological sample may be at least one selected from the group consisting of blood, whole blood, serum, urine, saliva, tissue, cell, organ, bone marrow, fine needle aspirate, core needle biopsy sample, and vacuum aspiration biopsy sample, but is not limited thereto.

In another embodiment of the present invention, if the method for providing information is for the diagnosis of aortic stenosis, the control group may be a biological sample separated from a normal individual, but is not limited thereto.

In yet another embodiment of the present invention, if the method for providing information is for predicting the prognosis of aortic stenosis, the control group may be a biological sample separated from either a normal individual or a patient with aortic stenosis, but is not limited thereto.

In yet another embodiment of the present invention, the prognosis may be at least one selected from the group consisting of occurrence, severity, progression, recurrence, disease-free survival, degeneration of the aortic valve, and the occurrence of complications related to aortic stenosis, but is not limited thereto.

In yet another embodiment of the present invention, the complication may be at least one selected from the group consisting of hypertension, rheumatoid arthritis, and interstitial lung disease, but is not limited thereto.

In yet another embodiment of the present invention, the measurement of the level of the anti-citrullinated peptide antibodies may be by at least one method selected from the group consisting of Western blot, fluorescence enzyme immunoassay (FEIA), enzyme-linked immunosorbent assay (ELISA), chemiluminescent microparticle immunoassay (CMIA), radioimmunoassay, radial immunodiffusion, Ouchterlony immunodiffusion, Rocket immunoelectrophoresis, immunostaining, immunoprecipitation analysis, complement fixation analysis, mass spectrometry, fluorescence-activated cell sorting (FACS), and protein chip, but is not limited thereto.

In yet another embodiment of the present invention, the aortic stenosis may be accompanied by calcification of the aortic valve, but is not limited thereto.

In addition, the present invention provides a screening method for a therapeutic substance of aortic stenosis, comprising the steps of:
(S1) treating a biological sample separated from an aortic stenosis patient with a test substance;
(S2) measuring at least one selected from the group consisting of the level of anti-citrullinated peptide antibodies, the level of citrullinated proteins, and the activity degree of citrullinated proteins in the sample treated with the test substance and a control sample not treated; and
(S3) selecting a test substance that reduces the level of the anti-citrullinated peptide antibodies, the level of citrullinated proteins, or the activity degree of citrullinated proteins compared to the control sample as a therapeutic substance for aortic stenosis.

Also, the present invention provides a composition for diagnosing or predicting the prognosis of aortic stenosis, comprising as an active ingredient an agent for measuring the level of anti-citrullinated peptide antibodies.

In one embodiment of the present invention, the agent for measuring the level of anti-citrullinated peptide antibodies may be a protein or aptamer specific to the anti-citrullinated peptide antibodies, but is not limited thereto.

Moreover, the present invention provides a kit for diagnosing or predicting the prognosis of aortic stenosis, comprising a composition comprising as an active ingredient an agent for measuring the level of anti-citrullinated peptide antibodies according to the present invention.

In one embodiment of the present invention, the kit may further comprise a manual describing the method for providing information according to the present invention, but is not limited thereto.

Furthermore, the present invention provides a method for diagnosing or predicting the prognosis of aortic stenosis, comprising the steps of:
(S1) measuring one or more selected from the group consisting of the level of anti-citrullinated peptide antibodies, the level of citrullinated proteins, and the activity degree of citrullinated proteins from a biological sample separated from a test subject; and
(S2) diagnosing aortic stenosis or determining that the prognosis of aortic stenosis is unfavorable if the level of anti-citrullinated peptide antibodies, the level of citrullinated proteins, and/or the activity degree of citrullinated proteins measured in step (S1) is increased compared to the control group.

Additionally, the present invention provides a use of anti-citrullinated peptide antibodies for diagnosing or predicting the prognosis of aortic stenosis.

Moreover, the present invention provides a use of an agent for measuring the level of anti-citrullinated peptide antibodies for diagnosing or predicting the prognosis of aortic stenosis.

Furthermore, the present invention provides a use of an agent for measuring the level of anti-citrullinated peptide antibodies for the production of a drug for diagnosing or predicting the prognosis of aortic stenosis.

### Advantageous Effects

The present invention relates to a method for providing information for diagnosing and/or predicting the prognosis of aortic stenosis. Based on a retrospective observation of about 700 patients who underwent both echocardiography and anti-citrullinated peptide antibody tests, it was confirmed that an increase in the level of anti-citrullinated peptide antibodies is associated with the occurrence and progression of aortic stenosis. Specifically, the inventors have verified that by measuring the level of anti-citrullinated peptide antibodies, not only can the occurrence of aortic stenosis be accurately diagnosed at an early stage, but it is also possible to predict the degeneration degree of the aortic valve, severity, and potential complications such as hypertension, rheumatoid arthritis, and interstitial lung disease. Therefore, anti-citrullinated peptide antibodies are expected to be utilized as a more effective biomarker for diagnosing, predicting prognosis, and treating aortic stenosis.

### BRIEF DESCRIPTION OF THE DRAWINGS

FIG. 1 is a schematic diagram illustrating the generation process of citrullinated protein by peptidylarginine deiminase (PAD) contributing to the onset of aortic stenosis.

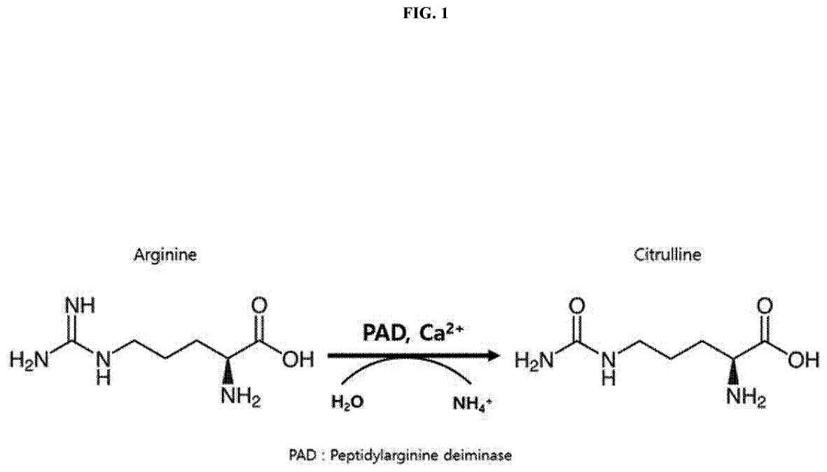

FIG. 2 is a research overview related to the present invention. A total of 678 test subjects who received both transthoracic echocardiography and anti-citrullinated peptide antibody (anti-CCP antibody) tests were studied, and 331 patients subsequently underwent transthoracic echocardiography.

FIG. 3A represents results confirming citrullinated protein in normal aortic valve tissue and aortic valve tissue of patients with aortic stenosis (AS) using immunohistochemistry.

FIG. 3B represents a comparison of quantified proportions of stained citrullinated proteins in photos of immunohistochemically stained normal aortic valve tissue or AS patient's aortic valve tissue.

FIG. 4 represents results confirming the level of citrullinated protein in normal aortic valve tissue and in aortic valve tissue of AS patients using Western blot.

FIG. 5 represents anti-citrullinated peptide antibody titer levels, degree of aortic valve degeneration, and progression of aortic valve stenosis by anti-citrullinated peptide antibody titer group.

FIG. 6 represents independent factors for predicting aortic stenosis.

### MODES OF THE INVENTION

The inventors of the present invention have found, based on the results of a retrospective observational study on patients who underwent both echocardiography and tests for specific anti-citrullinated peptide antibodies, that an increase in the titer of anti-citrullinated peptide antibodies may act as an independent predictive factor for the progression of aortic stenosis, and thus completed the present invention.

Specifically, in one embodiment of the present invention, upon comparing the levels of citrullinated protein in degenerated aortic valve tissue of patients with aortic stenosis and in normal aortic valve tissue, it was found that the level of citrullinated protein increased in the aortic valve tissue of patients with aortic stenosis, confirming the relationship between aortic stenosis and citrullinated peptide (Example 1).

In another embodiment of the present invention, after examining the clinical characteristics of test subjects based on the degeneration of the aortic valve, it was found that patients with degenerated aortic valves had not only coronary artery diseases and hypertension but also a higher positive rate of anti-citrullinated peptide antibodies (Example 2).

In yet another embodiment of the present invention, upon comparing patient characteristics by anti-citrullinated peptide antibody titer groups (negative, low-positive, high-positive), it was found that the high-positive group for anti-citrullinated peptide antibodies had higher rates of complications such as hypertension, rheumatoid arthritis, and interstitial lung disease, and both positive groups (low-positive, high-positive) had a higher rate of aortic valve degeneration (Example 3-1).

In yet another embodiment of the present invention, when comparing clinical outcomes based on the titer of anti-citrullinated peptide antibodies, it was found that the high-positive group and low-positive group had a higher progression rate of aortic stenosis compared to the negative group (Example 3-2).

In yet another embodiment of the present invention, upon confirming the association between the titer of anti-citrullinated peptide antibodies and aortic stenosis through univariate and multivariate analyses, it was found that the titer of anti-citrullinated peptide antibodies may function as an independent predictive factor for the progression of aortic stenosis (Example 4).

Hereinafter, the present invention is described in detail.

The present invention provides a method for providing information for diagnosing or predicting the prognosis of aortic stenosis, comprising the steps of:
(S1) measuring at least one selected from the group consisting of the level of anti-citrullinated peptide antibodies, the level of citrullinated protein, and the activity degree of citrullinated protein from a biological sample separated from a test subject; and
(S2) confirming whether the level of the anti-citrullinated peptide antibodies, the level of citrullinated protein, or the activity degree of citrullinated protein measured in step (S1) is increased compared to a control group.

In the present invention, the method for providing information may further comprise a step of diagnosing aortic stenosis or determining that the prognosis of aortic stenosis is unfavorable if the level of anti-citrullinated peptide antibodies, the level of citrullinated proteins, and/or the activity degree of citrullinated proteins measured in step (S1) is increased compared to the control group.

In the present invention, aortic stenosis (AS) refers to a condition where the aortic valve, a heart valve located at the site where blood flows out from the left ventricle to the aorta, does not open well when the left ventricle contracts. Patients with aortic stenosis experience chest pain, syncope, and dyspnea because the blood does not flow sufficiently from the left ventricle to the aorta. In the present invention, aortic stenosis is characterized by degenerative changes of the aortic valve, specifically lipid deposition and/or inflammation of the aortic valve, and preferably, may be characterized by fibrosis and calcification of the aortic valve. Because aortic stenosis causes impairment of systemic blood flow, it may lead to excessive increase in cardiac contractility, resulting in cardiac hypertrophy, and if excessive cardiac contraction persists, the contractility of the heart muscle may decrease, leading to symptoms of heart failure.

In the present invention, the terms “citrullinated peptide” or “citrullinated protein” refer to peptides that have undergone citrullination by the enzyme protein arginine deaminase (PAD). Citrullination is one of the post-translational modifications of proteins, occurring by deamination of peptidyl-arginine by the PAD enzyme. Citrullination often occurs in several chronic inflammatory diseases and is known to be related to immunological mechanisms associated with inflammation. The citrullinated peptide according to the present invention may comprise any peptide containing Cit-Gly motifs without limitation. Preferably, the citrullinated peptide comprises cyclic citrullinated peptide (CCP), and synthetic CCP may also be included. For example, the citrullinated peptide according to the present invention may preferably include citrullinated pro-filaggrin peptide, filaggrin protein, vimentin, mutated vimentin, fibrinogen, ovalbumin, EBNA-1, EBNA-2, proteoglycan, collagen, enolase, alpha-enolase, etc., but is not limited thereto. The aforementioned peptides and proteins are known, and the sequences of these peptides and proteins can be checked in public protein databases such as Uniprot (https://www.uniprot.org). Some of the sequences of citrullinated peptides are summarized in the table below (in the sequence, “X” indicates the citrullination site).

[Sequences of Citrullinated Peptides]

| | | SEQ |
|---|---|---|
| | | ID |
| Protein Name | Sequence | NO |
| Pro-filaggrin (1) | HQCHQEST-X-GRSRGRCSGS | 1 |
| Pro-filaggrin (2) | SHQEST-X-GRSRGRS | 2 |
| Fibrinogen | DHEGTHSTK-X-GHAKSPVRD | 3 |
| Ovalbumin (1) | TRTQINKVV-X-GDKLPGFGDS | 4 |
| Ovalbumin (2) | VESTQNGII-X-GVLQPSSVDS | 5 |
| EBNA-1 (1) | GGR-X-GRGERARGGSERAR | 6 |
| EBNA-1 (2) | GGRGRGRGERA-X-GGSERAR | 7 |
| EBNA-1 (3) | A-X-GGSRERARGGRGRGEKR | 8 |
| EBNA-1 (4) | ARGGSRERARGGRG-X-GEKR | 9 |
| EBNA-1 (5) | GGGKSTLYSNLR-X-GTALAIPQ | 10 |
| Proteoglycan | PQASVPLRLT-X-GRASPISRAQ | 11 |
| α-Enolase | CKIHA-X-EIFDS-X-GNPTVEC | 12 |
| Vimentin | GRVYAT-X-SSAVR | 13 |

In the specification used herein, the term “antibodies” refers to specific protein molecules directed to an antigenic site. The antibodies of the present invention comprise portions of the entire antibody having antigen-antibody binding ability, and all types of immunoglobulin antibodies that specifically bind to the target protein are comprised without limitation. For example, it comprises not only a complete form of the antibody having two full-length light chains and two full-length heavy chains but also functional fragments of the antibody molecule, i.e., Fab, F(ab′), F(ab′)2, and Fv, which have antigen binding functions.

Generally, antibodies are produced when foreign substances invade the body, recognizing the foreign substance as an antigen and neutralizing it, but in the case of “autoantibodies,” they are known to specifically bind recognizing one's own body components as antigens. That is, an autoantibody is an antibody produced against one's own body components due to an abnormality in the immune system, and excessive production of autoantibodies is known to induce autoimmune diseases such as rheumatoid arthritis, lupus, and antiphospholipid syndrome.

According to the present invention, “anti-citrullinated peptide antibodies” refer to autoantibodies that can specifically bind to citrullinated peptides in the body, i.e., all autoantibodies that recognize peptides containing Cit-Gly motifs as an antigen (epitope). That is, anti-citrullinated peptide antibodies according to the present invention are not limited in type and comprise any that can specifically bind to citrullinated peptides. Throughout this specification, “anti-citrullinated peptide antibodies” may be interchangeably used with anti-CCP (cyclic citrullinated peptide) antibodies, anti-CCP antibodies (Ab).

In the present invention, “diagnosis” refers to determining the susceptibility of a subject to a specific disease or condition, determining whether the subject currently has a specific disease or condition, determining the prognosis of a subject who has a specific disease or condition, or comprises therametrics (e.g., monitoring the condition of a subject to provide information on therapeutic efficacy).

In the present invention, “prognosis” refers to predictions regarding the onset, progression, recovery, recurrence, and drug resistance of a disease, meaning a forecast or preliminary evaluation. More preferably, in the present invention, “prognosis” may refer to one or more selected from the group consisting of the onset, severity, progression, recurrence, disease-free survival, degeneration of the aortic valve, and occurrence of complications of aortic stenosis. Thus, determining a good prognosis for aortic stenosis in the present invention may mean that the possibility of onset, severity, potential for progression, potential for recurrence, potential for degeneration of the aortic valve, and/or potential for complications is lower than normal individuals or other patients with aortic stenosis, and the expected survival period (e.g., disease-free survival period) is longer. Conversely, determining an unfavorable prognosis for aortic stenosis in the present invention may mean that the possibility of onset, severity, potential for progression, potential for recurrence, potential for degeneration of the aortic valve, and/or potential for complications is higher than normal individuals or other patients with aortic stenosis, and the expected survival period (e.g., disease-free survival period) is shorter. As mentioned above, “degeneration of the aortic valve” includes lipid deposition, inflammation, fibrosis, and/or calcification in the aortic valve.

In the present invention, the “progression” of aortic stenosis may refer to the worsening of symptoms, namely, the intensification of severity. Specifically, severity may be classified as none, mild, moderate, or severe, and in the present invention, the “progression” of aortic stenosis may refer to the severity worsening by at least one grade.

Moreover, in the present invention, the “complication” may comprise, without limitation, any disease associated with aortic stenosis or a known disease that induces aortic stenosis. That is, it may include any disease known to occur concurrently with aortic stenosis or before or after it. Preferably, the complications may comprise heart failure, endocarditis, syncope, angina, death, death from cardiovascular diseases, aortic valve replacement, aortic valve dysfunction, mitral valve dysfunction, mitral stenosis, rheumatoid arthritis, hypertension, diabetes, coronary artery disease, chronic kidney disease, cerebrovascular accident, and interstitial lung disease. Preferably, it may refer to rheumatoid arthritis, hypertension, and/or interstitial lung disease.

In the present invention, the “biological sample” may be used without limitation if it is collected from a test subject to diagnose or predict the prognosis of aortic stenosis, and preferably, it may include samples commonly used in anti-citrullinated peptide antibody tests. For instance, the biological sample may include one or more selected from the group consisting of blood, whole blood, serum, urine, saliva, tissue, cell, organ, bone marrow, fine needle aspirate, core needle biopsy sample, and vacuum aspiration biopsy sample, but is not limited thereto.

The biological sample may be preprocessed before being used for detection or diagnosis. For example, it may include homogenization, filtration, distillation, extraction, concentration, inactivation of interfering components, and addition of reagents. The sample may be prepared to enhance the detection sensitivity of protein markers, for instance, a sample obtained from a patient may be preprocessed using methods such as anion exchange chromatography, affinity chromatography, size exclusion chromatography, liquid chromatography, sequential extraction, or gel electrophoresis.

In the present invention, the “control” may refer to a biological sample isolated from a normal individual or a patient with aortic stenosis. Specifically, if the method for providing information according to the present invention is for diagnosing aortic stenosis, the control may refer to a biological sample isolated from a normal individual, and if the method for providing information is for predicting the prognosis of aortic stenosis, the control may refer to a biological sample isolated from a normal individual or a patient with aortic stenosis. The term “normal individual” refers to a subject who has never had aortic stenosis or a healthy subject. In the present invention, the “test subject” may be a mammal including humans and may refer to a subject who is suffering or suspected to be suffering from aortic stenosis.

In this specification, the term “measurement” encompasses the action of measuring and confirming the presence (expression) of a targeted substance (in the case of the present invention, anti-citrullinated peptide antibodies), or measuring and confirming the changes in the level (expression level) of the targeted substance. Hence, measuring the expression level of the protein comprises measuring the presence of the expression (i.e., measuring whether the expression is present or not) or measuring the qualitative or quantitative changes in the level of the protein. Such measurement may be conducted without limitation, encompassing both qualitative and quantitative methods. The types of qualitative and quantitative methods for measuring protein levels are well known in the art, and the experimental methods described in this specification are included therein. Each method has its specific protein level comparison method known in the art. Thus, detecting the target protein includes detecting the presence of anti-citrullinated peptide antibodies and confirming the increase (up-regulation) or decrease (down-regulation) of the protein expression level.

Methods for measuring the level of anti-citrullinated peptide antibodies in the body, if based on protein measurement methods disclosed in the art, have no particular limitation but may be measured by methods such as Western blot, fluorescence enzyme immunoassay (FEIA), ELISA, chemiluminescent microparticle immunoassay (CMIA), radioimmunoassay, radial immunodiffusion, Ouchterlony immunodiffusion, Rocket immunoelectrophoresis, immunostaining, immunoprecipitation analysis, complement fixation analysis, mass spectrometry, FACS, and/or protein chips. Methods for measuring the level of anti-citrullinated peptide antibodies are particularly well known in the art, for example, treating a sample on a microplate coated with citrullinated protein and binding the anti-citrullinated peptide antibodies present in the sample to a specific citrullinated protein, and then treating it with a luminous enzyme (for example, HRP) bound anti-human IgG antibodies capable of binding to the anti-citrullinated peptide antibodies, and measuring the luminescence degree to measure the level of anti-citrullinated peptide antibodies in the sample.

Also, methods for measuring the level and/or activity degree of citrullinated proteins are disclosed in the art, and can be measured by methods such as Western blot, fluorescence enzyme immunoassay (FEIA), ELISA, chemiluminescent microparticle immunoassay (CMIA), radioimmunoassay, radial immunodiffusion, Ouchterlony immunodiffusion, Rocket immunoelectrophoresis, immunostaining, immunoprecipitation analysis, complement fixation analysis, mass spectrometry, FACS, and/or protein chips, but not limited thereto.

In this specification, the term “analysis” may preferably mean “measurement,” wherein the qualitative analysis may mean measuring and confirming the presence of the target substance, and the quantitative analysis may mean measuring and confirming the level (expression level) or amount of change of the target substance. In the present invention, analysis or measurement may be conducted without limitation, encompassing both qualitative and quantitative methods, and preferably, quantitative measurement may be performed.

In this specification, the term “level is increased” means that something previously undetected is now detected, or that the detected quantity is relatively greater than the normal level. Those skilled in the art can understand the opposite term of the above definition to have the opposite meaning.

For example, the level is “increased” means that the level in the experimental group is at least 1%, 2%, 3%, 4%, 5%, 10% or more, for example, 5%, 10%, 20%, 30%, 40%, or 50%, 60%, 70%, 80%, 90% or more higher, and/or 0.5 times, 1.1 times, 1.2 times, 1.4 times, 1.6 times, 1.8 times or more higher than that of the control group. Specifically, it may mean increased by 1 to 1.5 times, 1.5 to 2 times, 2 to 2.5 times, 2.5 to 3 times, 3 to 3.5 times, 3.5 to 4 times, 4 to 4.5 times, 4.5 to 5 times, 5 to 5.5 times, 5.5 to 6 times, 6 to 6.5 times, 6.5 to 7 times, 7 to 7.5 times, 7.5 to 8 times, 8 to 8.5 times, 8.5 to 9 times, 9 to 9.5 times, 9.5 to 10 times, or more than 10 times, but is not limited thereto.

Furthermore, the present invention provides a screening method for a therapeutic substance of aortic stenosis, comprising the steps of:
(S1) treating a biological sample separated from an aortic stenosis patient with a test substance;
(S2) measuring at least one selected from the group consisting of the level of anti-citrullinated peptide antibodies, the level of citrullinated proteins, and the activity degree of citrullinated proteins in the sample treated with the test substance and a control sample not treated; and
(S3) selecting a test substance that reduces the level of the anti-citrullinated peptide antibodies, the level of citrullinated proteins, or the activity degree of citrullinated proteins compared to the control sample as a therapeutic substance for aortic stenosis.

In the present invention, the term “test substance” refers to an unknown substance used in screening to determine whether it affects the level of anti-citrullinated peptide antibodies, the level of citrullinated protein, and/or the activity degree of citrullinated protein in a biological sample. The test substance may include small interference RNA (siRNA), short hairpin RNA (shRNA), microRNA (miRNA), ribozyme, DNAzyme, peptide nucleic acids (PNA), antisense oligonucleotides, recombinant plasmid, nanoparticles, protein, oligopeptide, antibodies, aptamer, natural extracts or chemical substances, but is not limited thereto. The term “control group” mentioned in the screening method may be a sample not treated with the test substance, but is not limited thereto.

Furthermore, the present invention provides a composition for diagnosing or predicting the prognosis of aortic stenosis, comprising as an active ingredient an agent for measuring the level of anti-citrullinated peptide antibodies. Also, the agent for measuring the level of anti-citrullinated peptide antibodies may be a protein or aptamer specific to the anti-citrullinated peptide antibodies. Preferably, the protein specific to the anti-citrullinated peptide antibodies may include any protein that can specifically bind to the anti-citrullinated peptide antibodies without limitation. More preferably, the protein specific to the anti-citrullinated peptide antibodies may be an antibody specific to the anti-citrullinated peptide antibodies.

In the present invention, “antibodies” specific to anti-citrullinated peptide antibodies refer to specific protein molecules directed against the antigenic site of the anti-citrullinated peptide antibodies. For the purpose of the present invention, an antibody as an agent for measuring the level of the anti-citrullinated peptide antibody refers to an antibody that specifically binds to the anti-citrullinated peptide antibody, and comprises polyclonal antibodies, monoclonal antibodies, and recombinant antibodies. Furthermore, if it has antigen-antibody binding activity, a part of the entire antibody is also comprised in the antibodies of the present invention, and all types of immunoglobulin antibodies that specifically bind to the anti-citrullinated peptide antibody are comprised. For example, it comprises not only a complete form of antibody with 2 full-length light chains and 2 full-length heavy chains but also functional fragments of the antibody molecule, i.e., Fab, F(ab′), F(ab′)2, and Fv with antigen-binding functions. Moreover, specific antibodies to the anti-citrullinated peptide antibody of the present invention also comprise special antibodies such as humanized antibodies and chimeric antibodies, and recombinant antibodies that may specifically bind to the protein (anti-citrullinated peptide antibody) of the present invention.

In this specification, the term “aptamer” refers to a substance that may specifically bind to an analyte in a sample and means a single-stranded nucleic acid (DNA, RNA, or modified nucleic acid) with a stable tertiary structure by itself, which may specifically confirm the presence of a target protein in the sample. The production of the aptamer may be carried out by determining the sequence of an oligonucleotide with selective and high binding force for the target protein to be confirmed according to the general production method of the aptamer, and then synthesizing it, and modifying the 5′ or 3′ end of the oligonucleotide to bind to the functional group of the aptamer chip, such as —SH, —COOH, —OH, or NH₂, but is not limited thereto.

The composition of the present invention comprising the antibody specific to the anti-citrullinated peptide antibody may further comprise an agent necessary for detecting the disclosed protein, and the protein's expression level in a biological sample (biological sample obtained from the test subject in the present invention) may be measured without limitation using this composition.

Also, the present invention provides a kit for diagnosing or predicting the prognosis of aortic stenosis, comprising the composition according to the present invention. Moreover, the kit may further include a manual describing the method for providing information for diagnosing or predicting the prognosis of aortic stenosis according to the present invention.

The kit of the present invention may include not only antibodies and/or aptamers recognizing the target protein as a marker but also one or more other component compositions, solutions, or devices suitable for the analysis method. For example, the kit according to the present invention may include a plate coated with citrullinated peptides, enzymes and/or substrates for luminescent reactions for detecting the binding of the anti-citrullinated peptide antibody to the citrullinated peptides, washing solutions, control group samples, etc., but is not limited thereto.

Furthermore, the present invention provides a method for preventing, improving, and/or treating aortic stenosis, comprising the steps of:
(S1) measuring at least one selected from the group consisting of the level of anti-citrullinated peptide antibodies from a biological sample separated from the test subject, the level of citrullinated protein, and the activity degree of the citrullinated protein;
(S2) confirming whether the level of the anti-citrullinated peptide antibodies, the level of citrullinated protein, or the activity degree of the citrullinated protein measured in the step (S1) is increased compared to a control group;
(S3) diagnosing as aortic stenosis or determining that the prognosis of aortic stenosis is unfavorable when the level of the anti-citrullinated peptide antibodies, the level of citrullinated protein, and/or the activity degree of the citrullinated protein measured in the step (S1) is increased compared to a control group; and
(S4) treating the aortic stenosis when diagnosed as aortic stenosis or determined to have an unfavorable prognosis in the step (S3).

In the present invention, the term “treatment” refers to all acts intended to improve or beneficially alter the targeted disease and the metabolic abnormalities associated with it. Such treatment may utilize methods such as chemotherapy, radiation therapy, surgical procedures, biological therapies, or the administration of antibiotics.

For example, the step (S4) may be achieved through valve repair surgery or valve replacement wherein the damaged valve is repaired or an artificial valve replacement is conducted. The artificial valve replacement means a surgery in which the damaged valve is removed and an artificial valve is inserted.

Alternatively, the step (S4) may be performed through a transcatheter aortic valve implantation (TAVI or TAVR), a non-surgical treatment targeting patients with high surgical risks, wherein a catheter accesses the aortic valve through the femoral artery or apex of the heart, and a tissue valve made of biological tissue is inserted.

In addition, the step (S4) may be performed using drugs known in the art to improve or treat aortic stenosis without limitation.

For instance, the term “chemotherapy” refers to the act of using chemicals to treat a specific disease (e.g., aortic stenosis) and the entirety of drugs used therein. Specific examples may include ACE inhibitors, antiarrhythmics, anticoagulants, beta-blockers, diuretics, and vasodilators.

The terms used in the present invention were chosen based on the commonly used terms considering the functions in the present invention, but these terms may vary according to the intentions of the skilled person in the art, precedents, emergence of new technologies, etc. Also, in certain cases, there are terms arbitrarily chosen by the applicant, and in such cases, their meanings will be detailed in the description of the corresponding invention. Therefore, the terms used in the present invention should be defined based on the meaning of the term and the content throughout the present invention, not just by the name of the term itself.

Throughout the specification of the present invention, when a part comprises a certain component, it means that, unless there is a particular indication to the contrary, it does not exclude other components and may include other components. The terms “about” and “substantially” used throughout the specification of the present invention are used in a sense close to or at the number when a specific manufacturing and material tolerance is presented, and are used to prevent unconscientious infringers from unfairly using the exact or absolute numbers mentioned for the understanding of the present invention.

Throughout the specification of the present invention, the term “combination of these” in the Makushi format means a mixture or combination of one or more selected from the group consisting of components listed in the Makushi format, meaning it includes one or more selected from the group consisting of the components.

Hereinafter, preferable examples are presented to aid in the understanding of the present invention. However, these examples are provided merely for easier understanding of the present invention, and the content of the present invention is not limited by these examples.

### EXPERIMENTAL EXAMPLE

### Experimental Example 1. Procurement of Human Aortic Valve Tissue

Aortic valve tissues from patients with or without aortic stenosis (AS) were obtained during aortic valve replacement or heart transplantation. All the patients clinically did not have rheumatoid arthritis. The example of the present invention adhered to the Declaration of Helsinki, and the Institutional Review Board (IRB) of Asan Medical Center in Seoul approved the research protocol for obtaining human samples, and written consent was obtained from all patients.

### Experimental Example 2. Isolation of Primary Aortic Valve Interstitial Cells

A single leaflet from the procured human aortic valve was cut into 4 to 5 pieces and then reacted with collagenase at 37° C. for 30 minutes to remove the aortic valve endothelial cells. Subsequently, additional culturing was done at 37° C. for 1.5 hours to obtain aortic valve interstitial cells (AVICs). The isolated human AVICs were cultured in DMEM/F12 containing 1% antibiotic-antimycotic and 10% FBS. Only cells with passages between 2 and 5 were used in experiments like Western blot.

### Experimental Example 3. Western Blot

The procured aortic valve and isolated AVICs were homogenized in 1×RIPA solution mixed with protease inhibitors, and the lysates were loaded onto SDS-PAGE gels. The separated proteins were transferred to a PVDF membrane. The PVDF membrane was then blocked at room temperature with 5% BSA for 1 hour. The pan-citrullinated protein antibody (abcam, ab6464, diluted 1:1000) and HRP-conjugated anti-rabbit IgG antibody were used for detection.

### Experimental Example 4. Immunohistochemistry

Frozen tissue blocks prepared through steps such as tissue fixation and sucrose sinking were cut to a thickness of 8 μm using a microtome. The cut tissues were washed with TBS containing 0.025% Triton X-100 and incubated at room temperature with blocking solution (Dako, X0909) for 30 minutes. After three washes, primary antibody (abcam, ab6464, diluted 1:1000) was applied to the tissue slides overnight at 4° C., while TBS was applied for the negative control. Tissues were then washed twice for 5 minutes each, incubated in peroxidase blocking solution for 10 minutes at room temperature to quench endogenous peroxidase activity. After two washes, the secondary antibody (Dako REAL Envision detection system, Dako, K5077) was applied for 30 minutes at room temperature. The antibody solution was then washed off, and the tissues were incubated in a mixed solution of DAB+ chromogen and substrate buffer (Dako, K5077) for 10 minutes. The reaction was terminated by immersing the tissues in water, followed by staining with hematoxylin for 2 minutes. The tissues were then dehydrated and cleared before being mounted and observed under a microscope.

### Experimental Example 5. Study Population

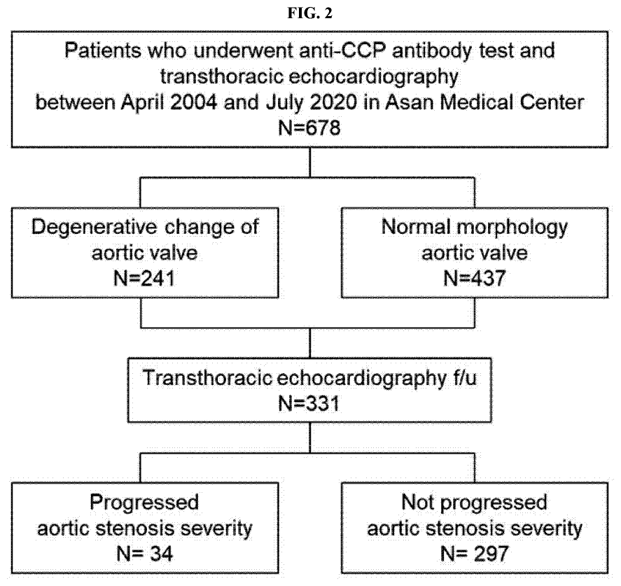

The study schematic is shown in FIG. 2. The present invention was conducted through a retrospective observational study at Asan Medical Center in Seoul, a tertiary referral hospital. Subjects aged 18 and above who underwent both echocardiography and anti-citrullinated peptide antibody (anti-CCP antibody) tests between April 2000 and July 2020 were enrolled. Results from transthoracic echocardiography conducted within six months before and after the anti-citrullinated peptide antibody test were included in the analysis. Among the test subjects, (i) patients who underwent aortic valve surgery or intervention before the anti-citrullinated peptide antibody test, (ii) patients with other serious valvular heart diseases (severity moderate or above), (iii) patients with rheumatic-valve diseases, and (iv) patients with a bicuspid aortic valve were excluded.

The research protocol received approval from the Institutional Review Board of Asan Medical Center in Seoul (IRB No.: 2019-1024). As it was a retrospective analysis, the need for prior consent was waived.

Demographic information, clinical diagnoses, and other experimental data were procured by reviewing the patient's charts. Not only the titer of anti-citrullinated peptide antibodies but also the erythrocyte sedimentation rate (ESR) and plasma C-reactive protein (CRP) were measured. Disease severity and the morphology of the aortic valve were assessed through 2D echocardiography and Doppler measurements like transaortic peak velocity.

### Experimental Example 6. Echocardiography Assessment

All patients (n=678) underwent echocardiography using M-mode, 2D, and color Doppler. Bicuspid and rheumatic valves stenosis were identified by their unique shapes. Aortic valve degeneration was defined when the leaflet of the aortic valve thickened or when at least one of the leaflets underwent calcification. The severity of aortic stenosis was classified into none, mild, moderate, or severe, integrating structural, Doppler, and quantitative parameters according to the guidelines of the American College of Cardiology/American Heart Association and the European Society of Cardiology. The LV ejection fraction was measured using the biplane Simpson volumetric method combining 4 and 2 chamber views. Echocardiography data for 331 patients (48.8%) were available for a follow-up of 12 months. For these subjects, the progression of aortic stenosis was assessed.

### Experimental Example 7. Anti-Citrullinated Peptide Antibody Assessment

Anti-citrullinated peptide antibodies in the subject's blood were measured using the fluorescence enzyme immunoassay (FEIA) (ELIA™) or the chemiluminescent microparticle immunoassay (CMIA) (Architect™). Levels of anti-citrullinated peptide antibodies were considered positive when the concentration was >10 U/mL for FEIA or ≥5.0 U/mL for CMIA, and levels below that were considered negative. To compare FEIA and CMIA plates, titers were classified as: negative (below the upper limit of normal, ULN), low-positive (above ULN and below median; FEIA=322.0 U/mL, CMIA=200.0 U/mL), and high-positive (median and above).

### Experimental Example 8. Research Results

The primary outcome was progression of aortic stenosis, wherein the progression of the stenosis was defined as an increase of at least one grade in the severity of AS in subsequent echocardiographic evaluations. The secondary outcomes include the aortic valve replacement rate, all-cause mortality, and cardiovascular cause mortality. The aortic valve replacements encompass both surgical and catheter aortic valve replacements.

### Experimental Example 9. Statistical Analysis

All data were presented as mean (± standard deviation) or median (interquartile range, IQR) for continuous variables, and as count (%) for categorical variables. Continuous variables were compared using the Student t-test or the Mann-Whitney U test, and categorical variables were compared using the Chi-squared or Fisher exact test. To evaluate the differences between anti-citrullinated peptide antibodies titer groups, analysis of variance (ANOVA) and Chi-squared tests were performed. To assess the association between the measured variables, binary logistic regression analysis was used for each variable individually for the progression of aortic stenosis, and univariate analysis was conducted. Independent predictive variables for the progression of aortic stenosis were measured through stepwise multivariable logistic regression, including variables with P values less than 0.10 in univariate analysis. All tests were conducted as two-sided, and P values less than 0.05 were considered statistically significant. All statistical analyses were conducted using the Statistical Package of Social Sciences version 22.0 (IBM Corp.).

### EXAMPLE

### Example 1. Confirmation of the Presence of Citrullinated Proteins in Human Aortic Valve Tissue

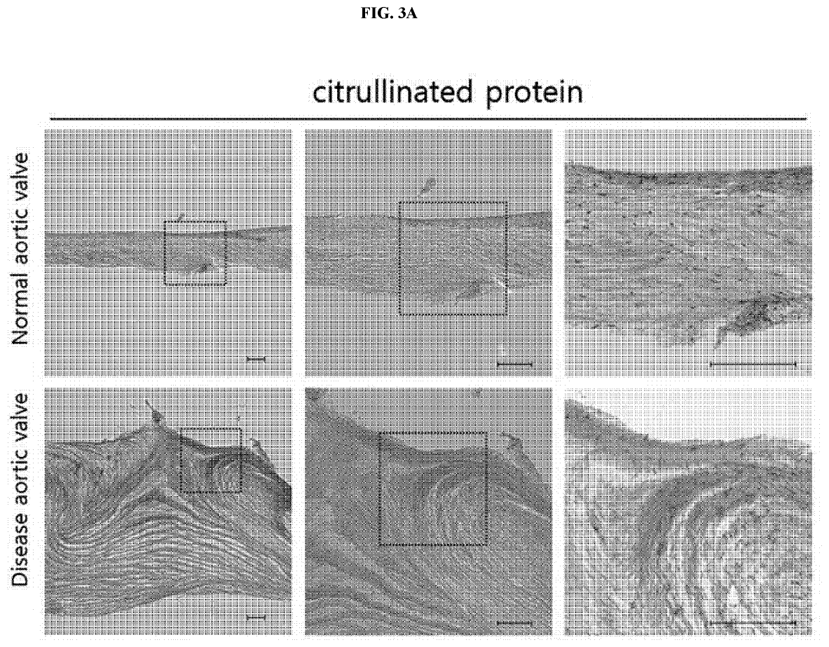

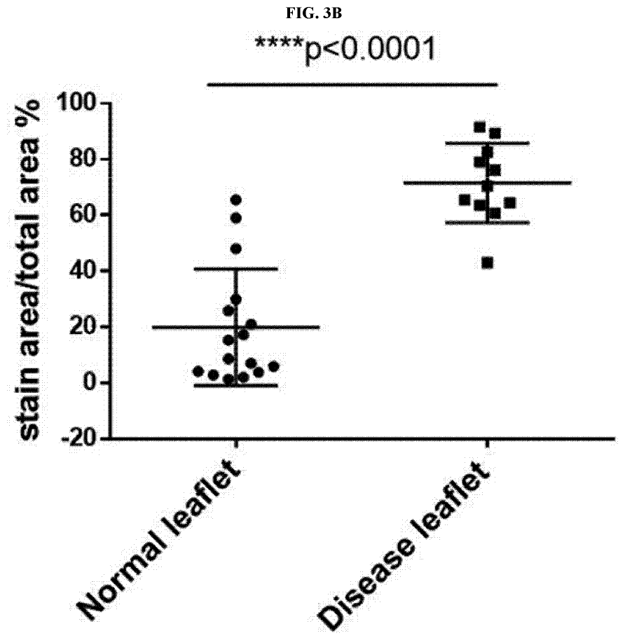

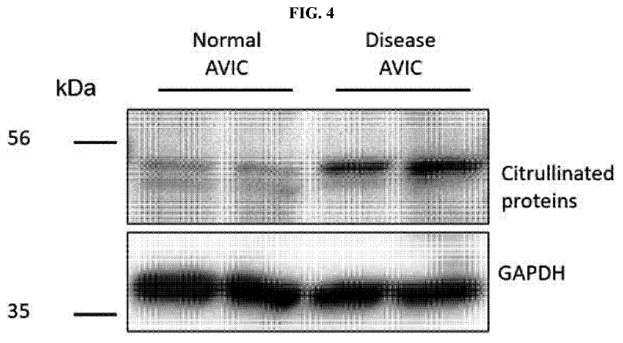

The inventors of the present invention confirmed the presence of citrullinated proteins in the aortic valve and AVIC using immunohistochemistry and Western blot. As a result of immunohistochemical analysis, as shown in FIG. 3A, it was confirmed that citrullinated proteins are present in the degenerative aortic valve tissue of patients with aortic stenosis, and after taking several high-magnification photos of normal aortic valve tissue (aortic valve tissue of patients who received a heart transplant) and the entire aortic valve tissue of patients with aortic stenosis, the proportion of stained citrullinated proteins in the photos was quantified and comparatively analyzed, confirming that the level of citrullinated proteins in the tissue of patients with aortic stenosis is higher than in normal aortic valve tissue (FIG. 3B). Also, through Western blot, it was confirmed that the level of citrullinated proteins increased in the aortic valve tissue of patients compared to the normal aortic valve tissue (FIG. 4). Such results imply that the inflammatory process of the aortic valve and the onset of aortic stenosis may be related to citrullinated peptides.

### Example 2. Characteristics of Test Subjects According to Degeneration of the Aortic Valve

Through eligibility screening, 678 patients with results of anti-citrullinated peptide antibodies and transthoracic echocardiography were identified (FIG. 2). Of the 678 patients, 254 showed positive results for anti-citrullinated peptide antibodies, and 424 showed negative results. Also, 241 patients appeared to have degenerative changes in the aortic valve, while the remaining 437 patients showed a normal aortic valve morphology. The baseline characteristics according to the presence or absence of degeneration of the aortic valve are shown in Table 1, and they were stratified by the degenerative changes of the aortic valve.

TABLE 1
Clinical Characteristics of Patients According to Aortic Valve Morphology

| | | | Degenerative | | Normal | | |
|---|---|---|---|---|---|---|---|
| | Total | | Change | | morphology | | P |
| Characteristics | (n = 678) | | (n = 241) | | (n = 437) | | value |
| Age | 62.1 ± 13.8 | | 69.8 ± 9.4 | | 57.9 ± 14.0 | | <0.001 |
| Male | 288 | (42.5) | 106 | (44.0) | 182 | (41.6) | 0.556 |
| Body Mass Index | 23.2 ± 3.9 | | 23.2 ± 3.9 | | 23.2 ± 4.0 | | 0.846 |
| (kg/m²) | | | | | | | |
| Anti-citrullinated | 254 | (37.5) | 103 | (42.7) | 151 | (34.6) | 0.035 |
| peptide Ab positive | | | | | | | |
| Hypertension | 204 | (30.1) | 98 | (40.7) | 106 | (24.3) | <0.001 |
| Diabetes mellitus | 141 | (20.8) | 62 | (25.7) | 79 | (18.1) | 0.019 |
| Coronary artery | 82 | (12.1) | 46 | (19.1) | 36 | (8.2) | <0.001 |
| disease | | | | | | | |
| Chronic kidney | 62 | (9.1) | 26 | (10.8) | 36 | (8.2) | 0.270 |
| disease | | | | | | | |
| Cerebrovascular | 24 | (3.5) | 12 | (5.0) | 12 | (2.7) | 0.132 |
| accident | | | | | | | |
| Atrial fibrillation/ | 50 | (7.4) | 24 | (10.0) | 26 | (5.9) | 0.056 |
| flutter | | | | | | | |
| Rheumatoid | 225 | (33.2) | 103 | (42.7) | 122 | (27.9) | <0.001 |
| arthritis | | | | | | | |
| Interstitial lung | 161 | (23.7) | 50 | (20.7) | 111 | (25.4) | 0.173 |
| disease | | | | | | | |
| Current or past | 208 | (30.7) | 77 | (32.0) | 131 | (30.0) | 0.594 |
| Smoking | | | | | | | |
| Baseline AV Vmax | 1.56 ± 0.71 | | 1.91 ± 1.03 | | 1.37 ± 0.30 | | <0.001 |
| (m/sec) | | | | | | | |
| Baseline LVEF (%) | 58.7 ± 10.9 | | 58.7 ± 10.4 | | 58.8 ± 11.1 | | 0.937 |
| Combined other | 78 | (11.5) | 32 | (13.3) | 46 | (10.5) | 0.282 |
| valve diseases | | | | | | | |

Values are represented as n (%) or mean (±SD).
Abbreviations: Ab, antibody; AV Vmax, peak aortic jet velocity; LVEF, left ventricle ejection fraction.

In general, patients with degenerated aortic valves often had higher ages and were frequently accompanied by hypertension, diabetes, and coronary artery disease. The positive rate of anti-citrullinated peptide antibodies was higher in patients with degenerated aortic valves than in patients with normal aortic valves (34.6% vs. 42.7%, p=0.035). Furthermore, the proportion of patients with rheumatoid arthritis was higher in the degenerated aortic valve group (27.9% vs. 42.7%, p<0.001). The peak aortic jet velocity was also higher in the degenerated aortic valve group.

### Example 3. Confirmation of the Characteristics of Test Subjects Based on the Level of Anti-citrullinated Peptide Antibodies

After more than a year of follow-up observations, echocardiography was performed on 331 patients, with a median follow-up period of 4.6 years (IQR: 2.0 to 8.0 years).

### Example 3-1. Comparison of Patient Characteristics by Titer Group of Anti-citrullinated Peptide Antibodies

To verify the correlation between the level of anti-citrullinated peptide antibodies and changes in the aortic valve, patient characteristics were compared based on the titer level of the anti-citrullinated peptide antibodies. The positive group for the anti-citrullinated peptide antibodies may be classified into the following two sub-groups: “low-positive”, which is above ULN and below the median (322.0 U/mL for FEIA and 200.0 U/mL for CMIA), and “high-positive”, which is at or above ULN. The characteristics based on the titer of the anti-citrullinated peptide antibodies were summarized in Table 2.

TABLE 2
Characteristics by Anti-Citrullinated Peptide Antibody Titer Group

| | Negative | | Low-positive | | High-positive | | P- |
|---|---|---|---|---|---|---|---|
| | (n = 424) | | (n = 138) | | (n = 116) | | value |
| Age | 59.4 ± 14.9 | | 65.6 ± 10.4 | | 67.8 ± 10.1 | | <0.001 |
| Male | 206 | (48.6) | 42 | (30.4) | 40 | (34.5) | <0.001 |
| Body Mass Index | 23.2 ± 3.9 | | 23.7 ± 3.9 | | 22.4 ± 3.9 | | 0.022 |
| (kg/m²) | | | | | | | |
| Hypertension | 113 | (26.7) | 48 | (34.8) | 43 | (37.1) | 0.014 |
| Diabetes mellitus | 94 | (22.2) | 24 | (17.4) | 23 | (19.8) | 0.392 |
| Coronary artery | 60 | (14.2) | 15 | (10.9) | 7 | (6.0) | 0.016 |
| disease | | | | | | | |
| Chronic kidney | 50 | (11.8) | 5 | (3.6) | 7 | (6.0) | 0.010 |
| disease | | | | | | | |
| Cerebrovascular | 17 | (4.0) | 3 | (2.2) | 4 | (3.4) | 0.571 |
| accident | | | | | | | |
| Atrial fibrillation/ | 35 | (8.3) | 8 | (5.8) | 7 | (6.0) | 0.312 |
| flutter | | | | | | | |
| Rheumatoid arthritis | 55 | (13.0) | 85 | (61.6) | 85 | (73.3) | <0.001 |
| Interstitial lung | 86 | (20.3) | 41 | (29.7) | 34 | (29.3) | 0.013 |
| disease | | | | | | | |
| Current or past | 144 | (34.0) | 34 | (24.6) | 30 | (25.9) | 0.035 |
| smoking | | | | | | | |
| Baseline AV Vmax | 1.54 ± 0.72 | | 1.55 ± 0.67 | | 1.65 ± 0.70 | | 0.279 |
| (m/sec) | | | | | | | |
| Baseline LVEF (%) | 57.6 ± 12.2 | | 60.8 ± 7.3 | | 60.5 ± 8.4 | | 0.002 |
| Combined other valve | 65 | (15.3) | 7 | (5.1) | 6 | (5.2) | <0.001 |
| diseases | | | | | | | |
| AV degenerative | 138 | (32.5) | 55 | (39.9) | 48 | (41.4) | 0.042 |
| change at baseline) | | | | | | | |
| Anti-citrullinated | 0.8 ± 1.9 | | 87.9 ± 79.5 | | 284.4 ± 68.0 | | <0.001 |
| peptide antibody titer | | | | | | | |
| (U/mL) | | | | | | | |
| CRP (mg/dL) | 2.8 | [0.5-8.4] | 1.6 | [0.4-5.5] | 1.7 | [0.5-5.8] | 0.055 |
| ESR (mm/hr) | 42.5 | [19.0-73.0] | 43.0 | [22.0-76.0] | 52.0 | [26.0-87.0] | 0.143 |

Values are represented as n (%) or mean (±SD) [IQR].
Abbreviations: AV Vmax, peak aortic jet velocity; LVEF, left ventricle ejection fraction; AV, aortic valve; Ab, antibody; CRP, C-reactive protein; ESR, erythrocyte sedimentation rate.

Compared to the negative group, the high-positive group was significantly older and had a higher prevalence of hypertension, rheumatoid arthritis, and interstitial lung disease (ILD). The prevalence of males, smokers, coronary artery disease, and chronic kidney disease was lower in the low-positive and high-positive groups compared to the negative group. However, the trend of degenerative changes of the aortic valve was higher in the positive groups compared to the negative group 715 (p=0.042). The levels of CRP and ESR showed no significant difference between the groups.

### 3-2. Clinical Outcome Analysis of Patients Based on the Titer Level of Anti-Citrullinated Peptide Antibodies

The clinical outcomes of patients based on the level of anti-citrullinated peptide antibodies are shown in Table 3. The median clinical follow-up period for all patients was 3.5 years (IQR: 1.1 to 7.5 years), and the median clinical follow-up period for patients who underwent echocardiography was 6.2 years (IQR: 3.4 to 9.8 years).

TABLE 3
Clinical Outcomes Based on the Titer of
Anti-Citrullinated Peptide Antibodies

| | Negative | | Low-positive | | High-positive | | P- |
|---|---|---|---|---|---|---|---|
| | (n = 424) | | (n = 138) | | (n = 116) | | value |
| Primary | | | | | | | |
| outcome | | | | | | | |
| TTE F/U | 5.5 ± 4.3 | | 6.1 ± 3.8 | | 5.4 ± 3.8 | | 0.578 |
| duration | | | | | | | |
| (years) | | | | | | | |
| AS | 19/227 | (8.4) | 7/62 | (11.3) | 8/42 | (19.0) | 0.041 |
| Progression* | | | | | | | |
| Secondary | | | | | | | |
| outcomes | | | | | | | |
| Clinical F/U | 4.7 ± 4.5 | | 4.9 ± 4.4 | | 4.5 ± 4.2 | | 0.761 |
| duration | | | | | | | |
| (years) | | | | | | | |
| Composite | 180 | (42.5) | 65 | (47.1) | 46 | (39.7) | 0.855 |
| of death, | | | | | | | |
| AVR | | | | | | | |
| All-cause | 171 | (40.3) | 62 | (44.9) | 44 | (37.9) | 0.906 |
| mortality | | | | | | | |
| CV cause | 14 | (9.3) | 4 | (6.5) | 4 | (9.3) | 0.822 |
| death | | | | | | | |
| Aortic valve | 20 | (4.7) | 6 | (4.3) | 5 | (4.3) | 0.826 |
| replacement | | | | | | | |

*Evaluated only in patients with available follow-up echocardiography results.
Values are represented as n/follow-up count (%), n (%), or mean (±SD).
Abbreviations: AS, aortic stenosis; F/U, follow-up; AVR, aortic valve replacement; CV, cardiovascular.

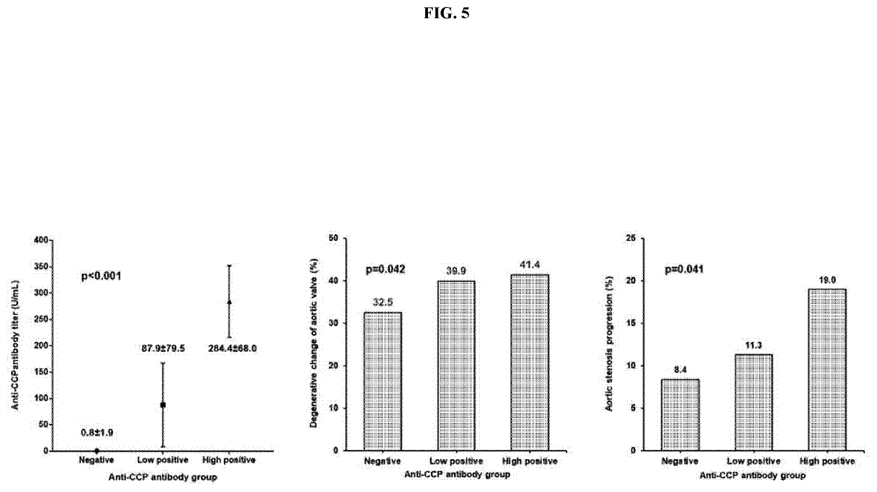

The progression of aortic stenosis occurred more frequently in the high-positive group compared to the low-positive or negative groups (high-positive vs. low-positive vs. negative=19.0% vs. 11.3% vs. 8.4%; p=0.041). There was no significant difference among the groups in all-cause mortality, cardiovascular cause mortality, aortic valve replacement, and the combined rate of all-cause mortality and aortic valve replacement. The titer levels of anti-citrullinated peptide antibodies, the proportion of degenerative aortic valves, and the progression degree of aortic stenosis by the anti-citrullinated peptide antibody titer group are shown in FIG. 5.

### Example 4. Confirmation of the Role of Anti-Citrullinated Peptide Antibodies Level as an Indicator for the Progression of Aortic Stenosis

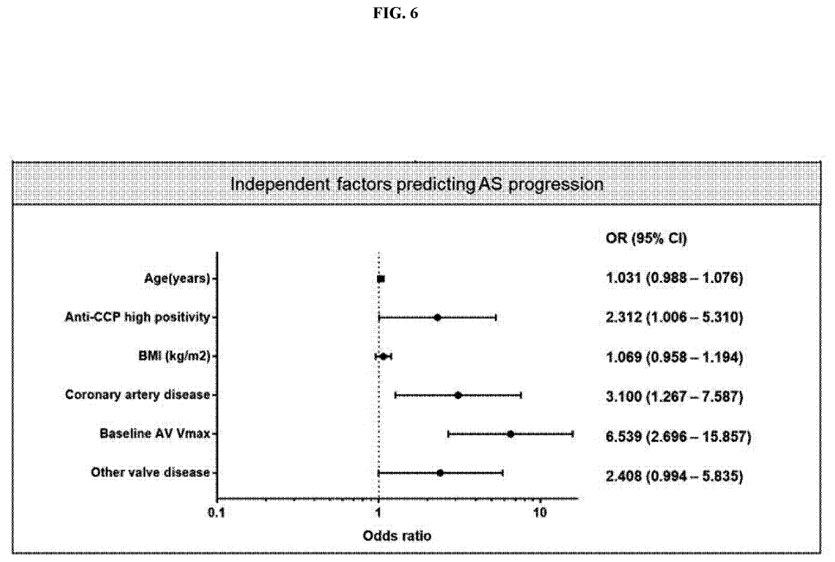

Univariate and multivariate analyses were performed on factors influencing the progression of aortic stenosis (Table 4 and FIG. 6).

TABLE 4
Predictive Factors of Aortic Valve Stenosis Progression

| | | | | Multivariate analysis | | |
|---|---|---|---|---|---|---|
| | Univariate analysis | | | Adjusted | | |
| | Odds | | | Odds | | |
| | ratio | 95% CI | P-value | ratio | 95% CI | P-value |
| Age | 1.056 | 1.018- | 0.004 | 1.031 | 0.988- | 0.157 |
| | | 1.095 | | | 1.076 | |
| Anti- | 1.932 | 0.931- | 0.077 | 2.312 | 1.006- | 0.048 |
| citrullinated | | 4.008 | | | 5.310 | |
| peptide | | | | | | |
| Ab high | | | | | | |
| positivity | | | | | | |
| Body Mass | 1.087 | 0.987- | 0.091 | 1.069 | 0.958- | 0.234 |
| Index (kg/m²) | | 1.197 | | | 1.194 | |
| Coronary artery | 3.955 | 1.804- | 0.001 | 3.100 | 1.267- | 0.013 |
| disease | | 8.670 | | | 7.587 | |
| Baseline AV | 8.994 | 3.713- | <0.001 | 6.539 | 2.696- | <0.001 |
| Vmax | | 21.786 | | | 15.857 | |
| Combined | 2.058 | 0.927- | 0.076 | 2.408 | 0.994- | 0.052 |
| other valve | | 4.571 | | | 5.835 | |
| diseases | | | | | | |

Abbreviation: Ab, antibody; AV max, peak aortic jet velocity.

From the multivariate analysis, a high positive amount of anti-citrullinated peptide antibodies was shown to be a significant factor in the progression of aortic stenosis (adjusted odds ratio (OR), 2.312; 95% CI, 1.006-5.310; p=0.048). Furthermore, combined coronary artery disease and baseline AV Vmax were identified as independent predictors of aortic stenosis progression (adjusted OR, 3.100; 95% CI, 1.267-7.587; p=0.010, and adjusted OR 6.539; 95% CI, 2.696-15.857; p<0.001). FIG. 6 clearly shows that a high titer of anti-citrullinated peptide antibodies is associated with aortic valve degeneration and may serve as an independent predictor of aortic stenosis progression.

As examined above, the inventors of the present invention confirmed the association between high levels of anti-citrullinated peptide antibodies, particularly high titers, and degenerative changes in the aortic valve. It was also confirmed that a positive amount of anti-citrullinated peptide antibodies may serve as an independent predictor for the progression of aortic stenosis. Therefore, the level of anti-citrullinated peptide antibodies according to the present invention is expected to be utilized as a biomarker for diagnosing aortic stenosis.

The description of the present invention as stated above is for illustrative purposes only, and a person having ordinary skill in the art to which the present invention pertains will understand that the technical idea or essential characteristics of the present invention can be easily modified into other specific forms without changing them. Therefore, the examples described above should be understood as illustrative in all aspects and not limited.

### INDUSTRIAL APPLICABILITY

The present invention is directed to providing methods for providing information for diagnosing and/or predicting the prognosis of aortic stenosis, and as a result of retrospective observation of approximately 700 patients who underwent both echocardiography and anti-citrullinated peptide antibody tests, it was confirmed that an increased level of anti-citrullinated peptide antibodies is associated with the onset and degree of progression of aortic stenosis. Specifically, the inventors of the present invention have confirmed that not only can the onset of aortic stenosis be accurately diagnosed early through the measurement of the level of anti-citrullinated peptide antibodies, but also the degree of valve degeneration, severity, and the possibility of complications such as hypertension, rheumatoid arthritis, and interstitial lung disease can be predicted. Therefore, anti-citrullinated peptide antibodies are expected to be utilized as a more effective biomarker for diagnosing, predicting prognosis, and treating aortic stenosis.

1. A method, comprising the steps of:
(S1) measuring at least one selected from the group consisting of a level of anti-citrullinated peptide antibodies, a level of citrullinated protein, and an activity degree of citrullinated protein from a biological sample separated from a test subject;
(S2) confirming whether the level of the anti-citrullinated peptide antibodies, the level of citrullinated protein, or the activity degree of citrullinated protein measured in step (S1) is increased compared to a control group;
(S3) diagnosing as aortic stenosis or determining that the prognosis of aortic stenosis is unfavorable when the level of the anti-citrullinated peptide antibodies, the level of citrullinated protein, and/or the activity degree of the citrullinated protein measured in the step (S1) is increased compared to a control group; and
(S4) treating the aortic stenosis with chemotherapy when diagnosed as aortic stenosis or determined to have an unfavorable prognosis in the step (S3).

2. The method of claim 1, wherein the biological sample is at least one selected from the group consisting of blood, whole blood, serum, urine, saliva, tissue, cell, organ, bone marrow, fine needle aspirate, core needle biopsy sample, and vacuum aspiration biopsy sample.

3. The method of claim 1, wherein when the method is for the diagnosis of aortic stenosis, the control group is a biological sample separated from a normal individual.

4. The method of claim 1, wherein when the method is for the prognosis prediction of aortic stenosis, the control group is a biological sample separated from either a normal individual or a patient with aortic stenosis.

5. The method of claim 1, wherein the prognosis is at least one selected from the group consisting of occurrence, severity, progression, recurrence, disease-free survival, degeneration of the aortic valve, and the occurrence of complications related to aortic stenosis.

6. The method of claim 5, wherein the complication is at least one selected from the group consisting of hypertension, rheumatoid arthritis, and interstitial lung disease.

7. The method of claim 1, wherein the measurement of the level of the anti-citrullinated peptide antibodies is by at least one method selected from the group consisting of Western blot, fluorescence enzyme immunoassay (FEIA), enzyme-linked immunosorbent assay (ELISA), chemiluminescent microparticle immunoassay (CMIA), radioimmunoassay, radial immunodiffusion, Ouchterlony immunodiffusion, Rocket immunoelectrophoresis, immunostaining, immunoprecipitation analysis, complement fixation analysis, mass spectrometry, fluorescence-activated cell sorting (FACS), and protein chip.

8. The method of claim 1, wherein the aortic stenosis is accompanied by calcification of the aortic valve.

9-13. (canceled)

14. A method, comprising the steps of:
(S1) measuring a level of anti-citrullinated peptide antibodies from a biological sample separated from a test subject;
(S2) confirming whether the level of the anti-citrullinated peptide antibodies measured in step (S1) is >10 U/mL for FEIA or >5.0 U/mL for CMIA;
(S3) diagnosing as aortic stenosis or determining that the prognosis of aortic stenosis is unfavorable when the level of the anti-citrullinated peptide antibodies measured in the step (S1) is >10 U/mL for FEIA or ≥5.0 U/mL for CMIA; and
(S4) treating the aortic stenosis with chemotherapy when diagnosed as aortic stenosis or determined to have an unfavorable prognosis in the step (S3).

15-16. (canceled)


SEQUENCE LISTING (13 sequences)

SEQ ID NO: 1 - protein, 21 aa, Artificial Sequence
  Synthetic Peptide
  MISC_FEATURE (9): citrullinated pro-filaggrin (1) (Xaa is citrulline)
HQCHQESTXGRSRGRCGRSGS

SEQ ID NO: 2 - protein, 14 aa, Artificial Sequence
  Synthetic Peptide
  MISC_FEATURE (7): citrullinated pro-filaggrin (2) (Xaa is citrulline)
SHQESTXGRSRGRS

SEQ ID NO: 3 - protein, 20 aa, Artificial Sequence
  Synthetic Peptide
  MISC_FEATURE (10): citrullinated fibrinogen (Xaa is citrulline)
DHEGTHSTKXGHAKSRPVRD

SEQ ID NO: 4 - protein, 20 aa, Artificial Sequence
  Synthetic Peptide
  MISC_FEATURE (10): citrullinated ovalbumin (1) (Xaa is citrulline)
TRTQINKVVXGDKLPGFGDS

SEQ ID NO: 5 - protein, 20 aa, Artificial Sequence
  Synthetic Peptide
  MISC_FEATURE (10): citrullinated ovalbumin (2) (Xaa is citrulline)
VESQTNGIIXGVLQPSSVDS

SEQ ID NO: 6 - protein, 20 aa, Artificial Sequence
  Synthetic Peptide
  MISC_FEATURE (4): citrullinated EBNA-1 (1) (Xaa is citrulline)
GGRXGRGRERARGGSRERAR

SEQ ID NO: 7 - protein, 20 aa, Artificial Sequence
  Synthetic Peptide
  MISC_FEATURE (12): citrullinated EBNA-1 (2) (Xaa is citrulline)
GGRRGRGRERAXGGSRERAR

SEQ ID NO: 8 - protein, 20 aa, Artificial Sequence
  Synthetic Peptide
  MISC_FEATURE (2): citrullinated EBNA-1 (3) (Xaa is citrulline)
AXGGSRERARGRGRGRGEKR

SEQ ID NO: 9 - protein, 20 aa, Artificial Sequence
  Synthetic Peptide
  MISC_FEATURE (16): citrullinated EBNA-1 (4) (Xaa is citrulline)
ARGGSRERARGRGRGXGEKR

SEQ ID NO: 10 - protein, 20 aa, Artificial Sequence
  Synthetic Peptide
  MISC_FEATURE (12): citrullinated EBNA-1 (5) (Xaa is citrulline)
GGSKTSLYNLRXGTALAIPQ

SEQ ID NO: 11 - protein, 21 aa, Artificial Sequence
  Synthetic Peptide
  MISC_FEATURE (11): citrullinated proteoglycan (Xaa is citrulline)
PQASVPLRLTXGSRAPISRAQ

SEQ ID NO: 12 - protein, 19 aa, Artificial Sequence
  Synthetic Peptide
  MISC_FEATURE (6): citrullinated alpha-enolase (Xaa is citrulline)
  MISC_FEATURE (12): citrullinated alpha-enolase (Xaa is citrulline)
CKIHAXEIFDSXGNPTVEC

SEQ ID NO: 13 - protein, 12 aa, Artificial Sequence
  Synthetic Peptide
  MISC_FEATURE (7): citrullinated vimentin (Xaa is citrulline)
GRVYATXSSAVR